(19) United States
(12) Patent Application Publication
(10) Pub. No.: US 2018/0059109 A1
(43) Pub. Date: Mar. 1, 2018

(54) METHOD OF DISTINGUISHING MESENCHYMAL STEM CELLS AND METHOD OF DETERMINING PURITY OF MESENCHYMAL STEM CELLS

(71) Applicant: Meridigen Biotech Co., Ltd., Taipei City (TW)

(72) Inventors: Chang-Yo HSUAN, Taipei City (TW); Willie LIN, Taipei City (TW); Yu-Chin SU, Taipei City (TW); Wei-Ting LIU, Taipei City (TW); Meng-Wei LI, Taipei City (TW); Mao-Kuang DU, Taipei City (TW)

(73) Assignee: Meridigen Biotech Co., Ltd., Taipei City (TW)

(21) Appl. No.: 15/371,607

(22) Filed: Dec. 7, 2016

Related U.S. Application Data
(60) Provisional application No. 62/381,934, filed on Aug. 31, 2016.

(51) Int. Cl. G01N 33/569; C12Q 1/68

(52) U.S. Cl. CPC G01N 33/56966; C12Q 2600/158; G01N 2333/70596; C12Q 1/6881

(57) ABSTRACT

A method of distinguishing mesenchymal stem cells (MSCs) from fibroblasts is provided. Also provided is a method of increasing a purity of mesenchymal stem cells (MSCs) population in a cell culture. The above-mentioned methods each comprise a step of sorting or isolating the cells by a marker of CD146 from a cell culture derived from a placenta-related tissue. Further provided is a method of assessing purity of mesenchymal stem cells (MSCs) in a cell culture derived from a placenta-related tissue, comprising determining the percentage of cells expressing CD146 in the culture.

### FIELD OF THE INVENTION

The present invention pertains to a method of distinguishing mesenchymal stem cells (MSCs) in a cell culture derived from a placenta-related tissue. The present invention also relates to a method of increasing the purity of MSC population in a cell culture derived from a placenta-related tissue. In another aspect, the invention pertains to a method of assessing purity of MSCs in a cell culture derived from a placenta-related tissue

### BACKGROUND OF THE INVENTION

Mesenchymal stem or stromal cells (MSCs) are multipotent cells of embryonic mesodermal origin, with a fibroblast-like morphology. These cells can differentiate into adipocytes, osteocytes, chondrocytes, neural lineage cells, and myocytes among other cell types depending on the stimuli and culture conditions. Although the plasticity of hMSCs and their role in tissue repair and regeneration have been extensively studied, it is their immunological trophic property that has gained the most interest recently [1-2]. Human mesenchymal stem cells have been isolated from a variety of tissues. The most frequently used source of MSCs is the bone marrow (BM). However, the isolation procedure is extremely invasive. To avoid the invasive isolation procedures, tissues such as human umbilical cord and placenta have been considered as good candidates since they are normally discarded after labor. The isolation of hMSCs from umbilical cord or placenta has proven to be efficient by previous studies [3].

MSCs are a subpopulation of a more complex cell composition of stromal cells contained in mesenchymal tissue. Due to the heterogeneous nature and the absence of known biomarkers specific for mesenchymal stem cells, it is a challenging task to define MSC phenotypes and characteristics [4-6]. The molecular components responsible for MSCs functionalities, in particular, those on the plasma membrane, remain largely unknown. In addition, lack of specific cell surface markers renders the cell culture at potential contamination risk with other cell types, in particular, those mature stromal cells such as fibroblasts, which, conversely, are abundant in mesenchymal tissue [4-6]. In the process of isolation of MSCs from placenta-derived tissues, non-MSCs, including fibroblasts, placenta-derived epithelial cells, and placenta-derived reticular cells, often co-exist with MSCs during the in vitro cultivation. In particular, fibroblast is the main source of contamination.

Fibroblasts are considered mature mesenchymal cells that are particularly abundant in the connective tissue. Therefore, these cells are the most frequent contaminating cell phenotype present in many cell culture systems. Not only is it difficult to apply techniques which successfully eliminate fibroblasts from a culture, it is also particularly complex to distinguish MSCs from fibroblasts in the same culture. Fibroblasts and MSCs have an extremely similar morphological appearance; they both proliferate well and share many identical cell surface markers [7-8]. MSC are currently defined as plastic adherent, multipotent fibroblast-like cells expressing CD73, CD90, CD105 and negative for the hematopoietic markers CD14, CD34 and CD45 by the International Society of Cellular Therapy (ISCT). However, these properties and markers are also shared by fibroblasts. The current definition suggested by ISCT is thus incapable of distinguishing MSC from generic fibroblasts. Until now, the best way to distinguish MSCs from fibroblasts is based on the analysis of the functional properties of these two kinds of cells; MSCs retain multipotent stemness and immunomodulation capacity, while fibroblasts seem more limited in both of these functional properties.

Since Friedenstein's pioneering work in identification of MSCs [48], there are no distinct differences in culture-derivation methodology, morphology, and gene expression signature that consistently and unequivocally distinguish ex vivo culture-expanded MSC from fibroblasts [9-12]. Presently, there is no accepted criterion or single cell-surface marker for separating the MSCs from fibroblasts. Due to the fact that fibroblast is the common contaminant cell population in MSC culture when derived from placenta, a novel surface protein as a biomarker to distinguish MSCs from fibroblasts is crucial to ensure the homogeneity of primary culture of placenta-derived MSCs.

CD146 (melanoma cell adhesion molecule, MCAM), a surface marker of vascular endothelial cells, functions by facilitating cell-cell interactions and angiogenesis. Recent research reveals that some MSCs express CD146 [13-14, 16], and the expression levels of CD146 are associated with their abilities of self-renewal, or abilities of differentiation into adipocytes and osteogenesis [13, 15]. Further, MSCs isolated from different tissues show different CD146 expression levels: about 40% of MSCs isolated from bone marrow express CD146, and its expression levels decrease as the passage number increases [13-15], while only less than 10% of MSCs isolated from adipose tissues express CD146 [14].

U.S. Pat. No. 9,470,685 discloses a method of distinguishing MSCs from fibroblasts using a marker EphA2 expressed on the MSCs.

### BRIEF SUMMARY OF THE INVENTION

It was unexpectedly found in the present invention that mesenchymal stem cells (MSCs) can be distinguished in a cell culture derived from a placenta-related tissue, based on their expression levels of a specific surface marker, CD146 (melanoma cell adhesion molecule, MCAM).

Accordingly, in one aspect, the present invention features a method of distinguishing mesenchymal stem cells (MSCs) from fibroblasts, comprising isolating the MSCs from the fibroblasts using a marker CD146 expressed on the MSCs so as to distinguish the MSCs from the fibroblasts, wherein the MSCs and fibroblasts are from a cell culture derived from a placenta-related tissue.

In certain embodiments of the present invention, the method further comprises the preliminary steps of: collecting the MSCs and the fibroblasts from the placenta-related tissue; and culturing the MSCs and the fibroblasts in a culture medium to prepare the cell culture.

In another aspect, the present invention provides a method of increasing a purity of mesenchymal stem cells (MSCs) population in a cell culture, comprising isolating and collecting MSCs using a marker CD146 expressed on the MSCs, wherein the MSCs are from a cell culture derived from a placenta-related tissue; and culturing the MSCs.

According to the present invention, the placenta-related tissue may be selected from the group consisting of amniotic membrane, chorionic disk, chorionic membrane, and umbilical cord. In one preferred embodiment, the placenta-related tissue is umbilical cord.

According to the present invention, the sorting step may be performed using a technique known or to be developed in the art, for example, an antibody-based or a nucleotide-based isolation method.

In certain embodiments of the present invention, the cells derived from a placenta-related tissue are cultured in a culture medium for MSC.

In a further aspect, the present invention provides a method of assessing purity of mesenchymal stem cells (MSCs) in a cell culture derived from a placenta-related tissue. Said method comprises the step of determining the percentage of cells expressing CD146 in the culture.

According to the present invention, the purity of MSCs has a positive correlation with the percentage of cells expressing CD146. In preferred embodiments of the present invention, the percentage of cells expressing CD146 is determined by a flow cytometry. In one embodiment, the purity of MSCs is calculated by the following equation: Y=SX+I, where Y is the purity (%) of MSCs, X is the percentage of cells expressing CD146, S is a value ranging from 1.129 to 1.283, and I is a value ranging from −28.676 to −17.964.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the results of flow cytometry analysis of mixed populations of MSCs and fibroblasts. MSCs derived from the umbilical cord from a donor were mixed with fibroblasts in different ratios. The results demonstrated that the percentage of CD146+ population detected by flow cytometry decreased proportionally in response to the increased fibroblast population. FB=fibroblasts.

FIG. 2 shows the results of flow cytometry analysis on CD146 expression of MSCs derived from umbilical cord at different passage numbers.

FIG. 3 shows the linear regression between the purity (%) of MSCs and the percentage of cells expressing CD146 in a cell culture derived from a placenta-related tissue.

FIG. 4A shows the CD146 expression level of CD146^{high} MSCs and CD146^{low} MSCs. FIG. 4B shows the results of adipogenesis differentiation from CD146^{high} MSCs and CD146^{low} MSCs.

FIG. 5 shows the results of PBMC proliferation assay.

FIG. 6 shows the levels of hepatocyte growth factor (HGF) in conditioned medium of CD146^{high} MSCs and CD146^{low} MSCs.

FIG. 7 shows the relative levels of cellular ROS, mitochondrial ROS and β-galactosidase (a senescence marker) in CD146^{high} MSCs and CD146^{low} MSCs.

### DETAILED DESCRIPTION OF THE INVENTION

The present invention is based on the unexpected finding that through a cell sorting by surface marker CD146, mesenchymal stem cells (MSCs) can be distinguished in a cell culture derived from a placenta-related tissue.

In one aspect, the present invention provides a method of distinguishing mesenchymal stem cells (MSCs) from fibroblasts. The method comprises isolating the MSCs from the fibroblasts using a marker CD146 expressed on the MSCs so as to distinguish the MSCs from the fibroblasts, wherein the MSCs and fibroblasts are from a cell culture derived from a placenta-related tissue.

In certain embodiments of the present invention, the method further comprises the preliminary steps of: collecting the MSCs and the fibroblasts from the placenta-related tissue; and culturing the MSCs and the fibroblasts in a culture medium to prepare the cell culture.

In another aspect, the present invention provides a method of increasing a purity of mesenchymal stem cells (MSCs) population in a cell culture, comprising isolating and collecting MSCs using a marker CD146 expressed on the MSCs, wherein the MSCs are from a cell culture derived from a placenta-related tissue; and culturing the MSCs.

In a still further aspect, the present invention provides a method of isolating a population of mesenchymal stem cells (MSCs), comprising isolating and collecting MSCs using a marker CD146 expressed on the MSCs, wherein the MSCs are from a cell culture derived from a placenta-related tissue, and the population of MSCs has a higher differentiation ability, elicits higher anti-inflammatory effects, has a lower degree of senescence, and/or secretes a higher amount of growth factors.

According to the present invention, the cells are freshly derived, obtained or collected from a placenta-related tissue following a protocol known in the art, for example, that of Shen et al. [21]. In certain preferred embodiments, the cells derived from a placenta-related tissue are then cultured in a culture medium for MSC. A standard medium for MSC comprises a minimal essential medium (with different versions of modification), fetal bovine serum (FBS), L-glutamine, and basic fibroblast growth factor (bFGF) [3, 16-20].

The placenta-related tissue may be selected from the group consisting of amniotic membrane, chorionic disk, chorionic membrane, and umbilical cord. In one preferred embodiment of the present invention, the placenta-related tissue is umbilical cord.

In carrying out the methods of the present invention, a cell culture derived from a placenta-related tissue is subjected to a cell sorting by CD146. The cell sorting may be performed through a technique known or to be developed in the art, for example, an antibody-based or a nucleotide-based isolation method. Preferably, the cell sorting is performed by an antibody-based magnetic cell sorting. For example, the MACS method (MACS® Technology, Miltenyi Biotec). In addition, the cell sorting may be preform through a flow cytometry method, e.g. an antibody-based or a nucleotide-based flow cytometry.

Further, it was unexpected found in the present invention that the purity of MSCs has a positive correlation with the percentage of cells expressing CD146.

Accordingly, the present invention in a further aspect provides a method of assessing purity of mesenchymal stem cells (MSCs) in a cell culture derived from a placenta-related tissue. Said method comprises the step of determining the percentage of cells expressing CD146 in the culture.

In preferred embodiments of the present invention, the percentage of cells expressing CD146 is determined by a flow cytometry.

In one embodiment, the purity of MSCs is calculated by the following equation: Y=SX+I, where Y is the purity (%) of MSCs, X is the percentage of cells expressing CD146, S is a value ranging from 1.129 to 1.283, and I is a value ranging from −28.676 to −17.964.

According to certain embodiments of the present invention, the population of MSCs isolated by a method of the present invention has a higher differentiation ability. In one embodiment, the population of MSCs has a higher adipogenesis ability.

According to certain embodiments of the present invention, the population of MSCs isolated by a method of the present invention elicits higher anti-inflammatory effects. In one embodiment, the population of MSCs exhibits a higher ability in inhibiting PBMC proliferation.

According to certain embodiments of the present invention, the population of MSCs isolated by a method of the present invention has a lower degree of senescence. In some embodiments, the population of MSCs expresses a lower level of cellular ROS, mitochondrial ROS, or beta-galactosidase.

According to certain embodiments of the present invention, the population of MSCs isolated by a method of the present invention secretes a higher amount of growth factors. In one embodiment, the population of MSCs secretes a higher amount of a hepatocyte growth factor (HGF).

It is to be understood that both the foregoing general description and the following detailed description are exemplary and explanatory only and are not restrictive of the invention.

The present invention is further illustrated by the following examples, which are provided for the purpose of demonstration rather than limitation.

### EXAMPLES

### Example 1: Flow Cytometry Analysis of Mixed Populations of MSCs and Fibroblast

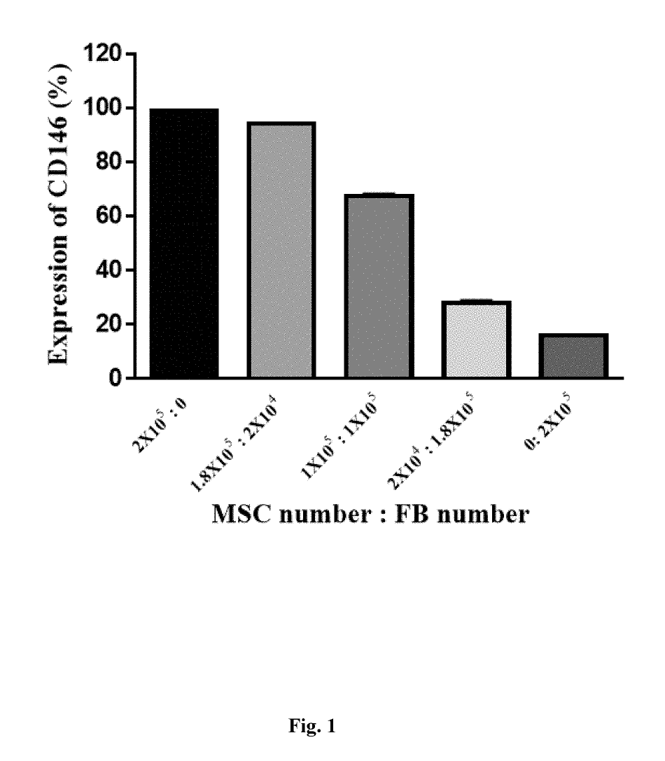

To demonstrate that CD146 could serve as a biomarker to separate placenta-derived MSCs from fibroblasts, MSCs derived from umbilical cord were mixed with fibroblasts in Eppendorf tubes by following ratios (MSC: fibroblasts in cell number): 2×10⁵:0, 1.8×10⁵:2×10⁴, 1×10⁵:1×10⁵, 2×10⁴:1.8×10⁵, or 0:2×10⁵. CD146+ population in each Eppendorf tube was then analyzed by flow cytometry. Referring to FIG. 1, the results shows that the percentage of CD146+ population detected by anti-CD146 antibodies (anti-human CD146-V450, BD biosciences) via flow cytometry decreased proportionally in relation to the increase of fibroblast population.

### Example 2: CD146 Expression Levels are Maintained as the Passage Number Increases

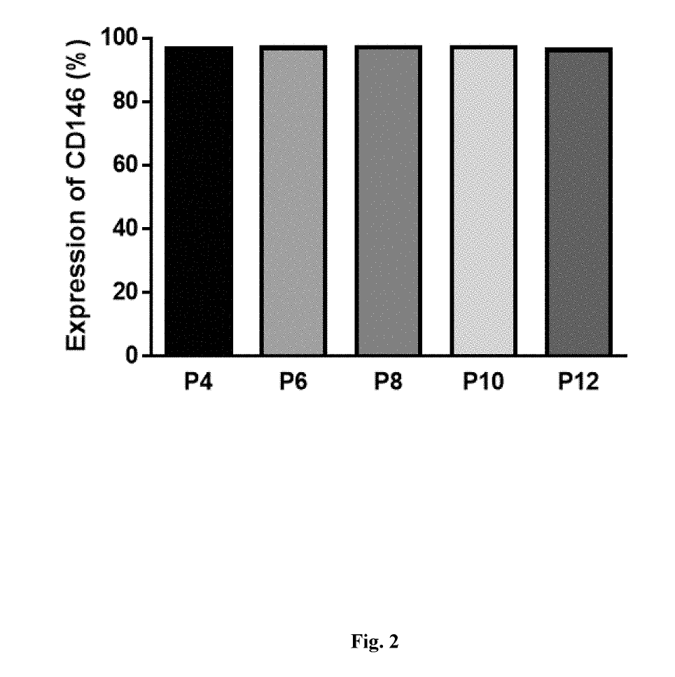

MSCs derived from umbilical cord were harvested at different passage numbers (passage numbers: 4, 6, 8, 10, and 12) and the expression levels of CD146 were analyzed by flow cytometry. The results show that CD146 expression levels are maintained along passages (FIG. 2).

### Example 3: Assessment of Purity of MSCs

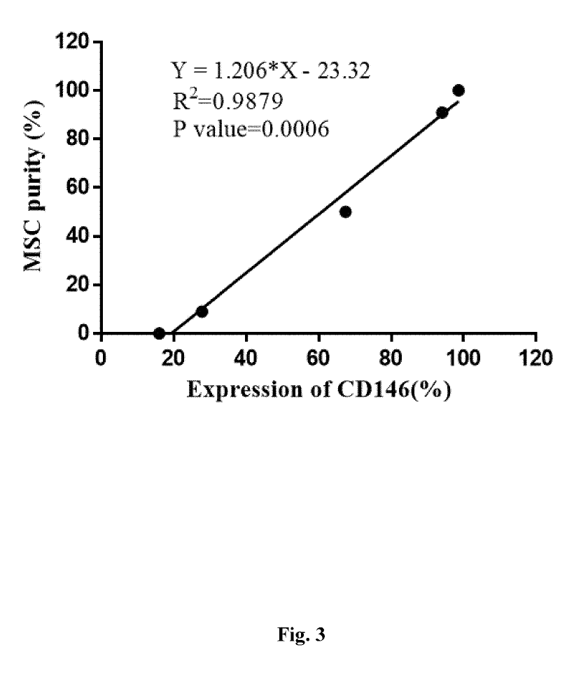

MSCs derived from umbilical cord were mixed with fibroblasts in Eppendorf tubes by following ratios (MSC: fibroblasts in cell number): 2×10⁵:0, 1.8×10⁵:2×10⁴, 1×10⁵:1×10⁵, 2×10⁴:1.8×10⁵, or 0:2×10⁵. CD146+ population in each Eppendorf tube was then analyzed by flow cytometry. Subsequently, the relationship between the purity (%) of MSCs and the percentage of cells expressing CD146 was analyzed by simple linear regression, results of which are shown in FIG. 3. The regression equation is Y=1.206X−23.32, where Y is the purity (%) of MSCs and X is the percentage of cells expressing CD146. The results demonstrated that there is a linear relationship between the expression level of CD146 and the purity of MSCs.

### Example 4: CD146^{high} MSCs Exhibits Higher Differentiation Ability than CD146^{low} MSCs

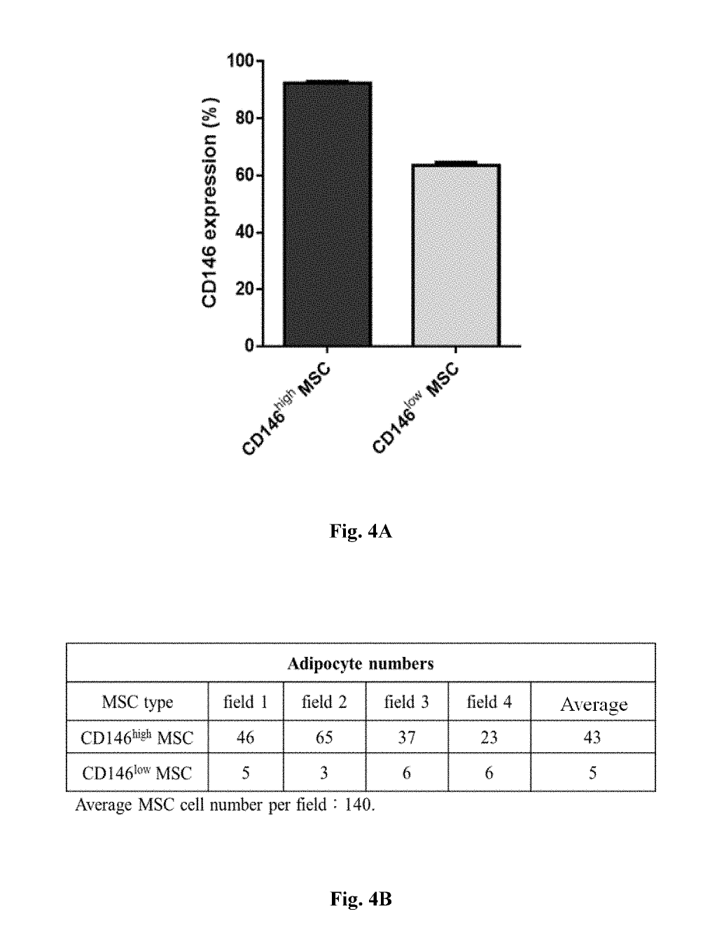

MSCs derived from different umbilical cords were examined for expression of CD146 using flow cytometry. Two groups of MSCs were obtained: CD146^{high} MSCs and CD146^{low} MSCs (FIG. 4A). 2×10⁵/well MSCs were seeded into 12-well plate, cultured overnight, and then cultured in adipocyte differentiation basal medium (InvitroGen) supplemented with adipogenesis supplement (InvitroGen). After culture for 14 days, the two groups of MSCs were fixed formalin, stained with Oil Red, and observed under a microscope to count the number of adipocytes. As shown in FIG. 4B, CD146^{high} MSCs exhibited higher adipogenesis ability.

### Example 5: CD146^{high} MSCs Elicits Higher Anti-Inflammatory Effects than CD146^{low} MSCs

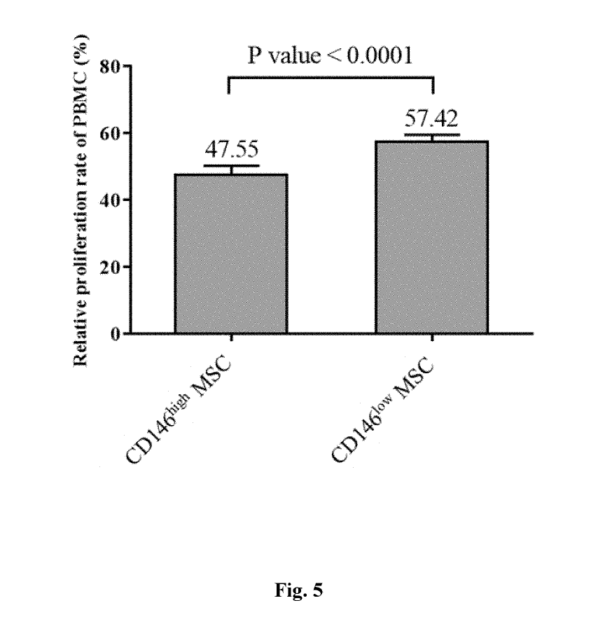

CD146^{high} MSCs and CD146^{low} MSCs were obtained as described in Example 4. The immune modulation capability of MSC was analyzed by PBMC proliferation assay. Carboxyfluorescein succinimidyl ester (CFSE) labeled PBMC is stimulated by phytohemagglutinin (PHA) and co-cultured with MSCs. The percentage of proliferated PBMCs was analyzed by FACS. The proliferation rate of PBMC was determined by assessing the reduction of the intensity of the fluorescent cell permeable dye CFSE. The results show that CD146^{high} MSCs were more effective in inhibiting PBMC proliferation than CD146^{low} MSCs (FIG. 5), suggesting that CD146^{high} MSCs elicit higher anti-inflammatory effects.

### Example 6: CD146^{high} MSCs Secretes a Higher Amount of Growth Factors than CD146^{low} MSCs

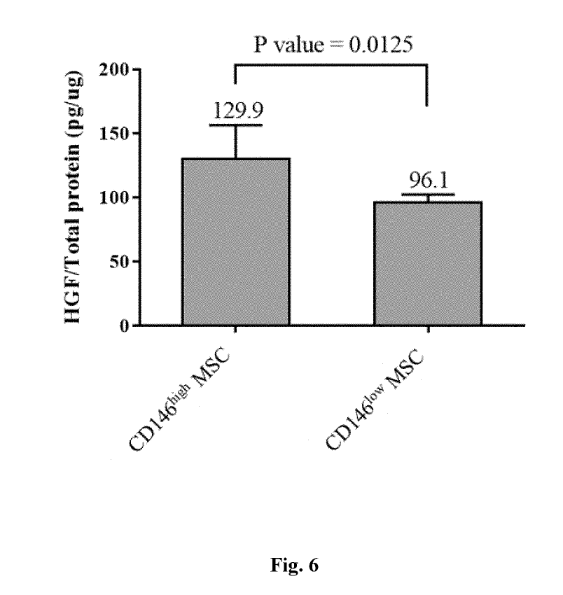

CD146^{high} MSCs and CD146^{low} MSCs were obtained as described in Example 4. Hepatocyte growth factor (HGF) level in conditioned medium from both groups was analyzed by ELISA and MSC total protein was analyzed by BCA assay. HGF level is expressed as a ratio of HGF amount to MSC total protein amount. As shown in FIG. 6, CD146^{high} MSCs secreted a significantly higher amount of HGF than CD146^{low} MSCs.

### Example 7: CD146^{high} MSCs have a Lower Degree of Senescence than CD146^{low} MSCs

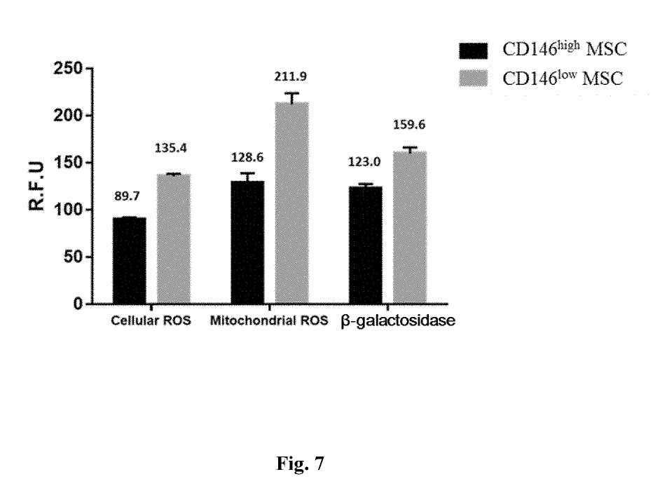

CD146^{high} MSCs and CD146^{low} MSCs were obtained as described in Example 4. Higher levels of cellular ROS, mitochondrial ROS and beta-galactosidase are considered to be related to senescence. In the present experiment, cellular ROS level and mitochondrial ROS level were determined using commercial kits (DCFDA of Sigma, and MitoSox of Invitrogen, respectively). The level of senescence-associated beta-galactosidase was determined using C12FDG (Molecular Probes). The results show that the levels of cellular ROS, mitochondrial ROS and beta-galactosidase in CD146^{high} MSCs were lower than in CD146^{low} MSCs (FIG. 7), suggesting that CD146^{high} MSCs have a lower degree of senescence than CD146^{low} MSCs.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### REFERENCES

[1] Stagg J et al., Curr Mol Med. 13(5):856-67 (2013).
[2] Casado J G et al., Stem Cell Rev. 9(2):184-9 (2013).
[3] Fukuchi Y et al., Stem Cells. 2004; 22(5):649-58.
[4] Javazon E H et al., Exp Hematol. 32(5):414-25 (2004).
[5] Nombela-Arrieta C et al., Nat Rev Mol Cell Biol. 12(2):126-31 (2011).
[6] Keating A., Cell Stem Cell. 10(6):709-16 (2012).
[7] Linge C et al., Exp Cell Res 1989, 185:519-528.
[8] Lorenz K et al., Exp Dermatol 2008, 17:925-932.
[9] Blasi et al., Vascular Cell 2011, 3:5.
[10] Covasa et al., Volume 36, Issue 5, Pages 642-654 (2008).
[11] Muzlifah A. Haniffa et al., Haematologica 94:258-263 (2009).
[12] Pittenger et al., Science Vol. 284 no. 5411 pp. 143-147 (1999).
[13] Lv F. J. et al., Stem Cells. 2014; 32(6):1408-19.
[14] Dmitrieva R. I. et al., Cell Cycle. 2012; 11(2)377-83.
[15] Halfon S. et al., Stem Cells Dev. 2011; 20(1):53-66.
[16] Miao Z et al., Cell Biol Int. 2006 September; 30(9):681-7.
[17] Malek A et al., J Stem Cells. 2011; 6(2):75-92.
[18] Cavallo C et al., J Cell Biochem. 2011 May; 112(5):1418-30. doi: 10.1002/jcb.23058.
[19] Shalini Vellasamy et al., World J Stem Cells. Jun. 26, 2012; 4(6): 53-61.
[20] Luan X et al., Tissue Cell. 2013 February; 45(1):32-8. doi: 10.1016/j.tice.2012.09.002. Epub 2012 Oct. 27.
[21] Shen et al., Taiwan J Obstet Gynecol. 2015 December; 54(6):749-56. doi: 10.1016/j.tjog.2015.10.012.

What is claimed is:

1. A method of distinguishing mesenchymal stem cells (MSCs) from fibroblasts, comprising:
isolating the MSCs from the fibroblasts using a marker CD146 expressed on the MSCs so as to distinguish the MSCs from the fibroblasts,
wherein the MSCs and fibroblasts are from a cell culture derived from a placenta-related tissue.

2. The method of claim 1, wherein the placenta-related tissue is selected from the group consisting of amniotic membrane, chorionic disk, chorionic membrane, and umbilical cord.

3. The method of claim 2, wherein the placenta-related tissue is umbilical cord.

4. The method of claim 1, wherein the isolating step is performed through an antibody-based or a nucleotide-based isolation method.

5. The method of claim 1, further comprising the preliminary steps of:
collecting the MSCs and the fibroblasts from the placenta-related tissue; and
culturing the MSCs and the fibroblasts in a culture medium to prepare the cell culture.

6. The method of claim 4, wherein the antibody-based isolation method is an antibody-based magnetic cell sorting or an antibody-based flow cytometry.

7. The method of claim 4, wherein the nucleotide-based isolation method is a nucleotide-based flow cytometry.

8. A method of increasing a purity of mesenchymal stem cells (MSCs) population in a cell culture, comprising:
isolating and collecting MSCs using a marker CD146 expressed on the MSCs, wherein the MSCs are from a cell culture derived from a placenta-related tissue; and
culturing the MSCs.

9. The method of claim 8, wherein the placenta-related tissue is selected from the group consisting of amniotic membrane, chorionic disk, chorionic membrane, and umbilical cord.

10. The method of claim 8, wherein the isolating step is performed through an antibody-based or a nucleotide-based isolation method.

11. The method of claim 8, wherein the cells derived from a placenta-related tissue are cultured in a culture medium for MSC.

12. The method of claim 10, wherein the antibody-based isolation method is an antibody-based magnetic cell sorting or an antibody-based flow cytometry.

13. The method of claim 10, wherein the nucleotide-based isolation method is a nucleotide-based flow cytometry.

14. A method of assessing a purity of mesenchymal stem cells (MSCs) in a cell culture derived from a placenta-related tissue, comprising determining the percentage of cells expressing CD146 in the culture.

15. The method of claim 14, wherein the percentage of cells expressing CD146 is determined by a flow cytometry.

16. The method of claim 14, wherein the purity of MSCs has a positive correlation with the percentage of cells expressing CD146.

17. The method of claim 14, wherein the purity of MSCs is calculated by the equation shown below:
Y=SX+I, where Y is the purity (%) of MSCs, X is the percentage of cells expressing CD146, S is a value ranging from 1.129 to 1.283, and I is a value ranging from −28.676 to −17.964.
